(19) United States
(12) Patent Application Publication
(10) Pub. No.: US 2026/0115173 A1
(43) Pub. Date: Apr. 30, 2026

(54) COMBINATION OF MITRAGYNINE AND NALTREXONE FOR SUBSTANCE USE DISORDERS

(71) Applicant: University of Houston System, Houston, TX (US)

(72) Inventors: Colin N. Haile, Houston, TX (US); Therese A. Kosten, Houston, TX (US); Joydip Das, Pearland, TX (US)

(73) Assignee: University of Houston System, Houston, TX (US)

(21) Appl. No.: 19/118,460

(22) PCT Filed: Oct. 5, 2023
(86) PCT No.: PCT/US23/34512

§ 371 Date: Apr. 4, 2025

Related U.S. Application Data
(60) Provisional application No. 63/414,152, filed on Oct. 7, 2022.

(51) Int. Cl. A61K 31/4375; A61K 31/485; A61P 25/32

(52) U.S. Cl. CPC A61K 31/4375; A61K 31/485; A61P 25/32

(57) ABSTRACT

A combination therapy for use in treatment of substance abuse disorders, including alcohol use disorder (AUD) and opioid use disorder (OUD), includes both mitragynine (MG) and naltrexone (NTX). NTX is understood to be a mu opioid antagonist that is expected to block MG's analgesic effects. However, combining MG and NTX therefore results in a unique, unexpected, and beneficial treatment that decreases alcohol self-administration to a greater extent than either alone.

This application claims priority to U.S. Provisional Patent Application Ser. No. 63/414,152, entitled “Combination Therapy for Substance Use Disorders,” filed Oct. 7, 2022, the entire contents of which are hereby incorporated by reference.

This invention was made with government support under grant 1R01 AA022414 awarded by the National Institutes of Health. The government has certain rights in the invention.

### BACKGROUND

This disclosure pertains to therapy for substance use disorders.

In the United States, excessive alcohol misuse is associated with thousands of deaths annually and billions in estimated losses. Worldwide alcohol consumption contributes to 3 million deaths per year and is the leading risk factor for mortality and disability in certain age groups (15-49 yrs). Alcohol use disorder (AUD) is a chronic relapsing disorder associated with compulsion to consume alcohol, inability to control alcohol drinking, negative affective states, and a withdrawal syndrome. Although there are FDA-approved pharmacotherapies for the treatment of AUD available, they have proven insufficient due to non-compliance and lack of efficacy, thus new innovative treatments are needed.

Generally, alcohol's reinforcing effects are mediated by the mesocorticolimbic system that includes neural circuitry emanating from the ventral tegmental area that projects to the nucleus accumbens (NAC) and pre-frontal cortex (CTX) and affects neurotransmitters including dopamine (DA), glutamate, gamma-aminobutyric acid (GABA), cannabinoids, and opioids. The opioid system in particular plays a pivotal role in alcohol reinforcement and consumption. Indeed, alcohol increases β-endorphin and DA in the NAC with the latter effect blocked by opioid antagonists whereas infusion of a mu opioid agonist into the NAC shell facilitates alcohol consumption. Consistent with preclinical data, alcohol induces opioid release in the NAC and orbitofrontal CTX in humans whereby the latter is positively correlated with subjective “high” in heavy drinkers. These data may partially explain why the mu opioid antagonist naltrexone (NTX) shows some efficacy for the treatment of AUD in humans.

While AUD is more prevalent in men than women, the gender gap in alcohol consumption is narrowing. In fact, the proportion of women who engage in binge drinking has increased in younger cohorts compared to older ones. Because frequent binge drinking during teenage years associates with an increased risk to develop AUDs later in life, it underscores the need to determine if pharmacotherapies for AUDs are effective in women who will make up a larger proportion of affected individuals in the near future.

### SUMMARY

The present disclosure relates generally to a combination therapy for use in treatment of substance use disorders, including alcohol use disorder (AUD) and opioid use disorder (OUD).

In particular, the present disclosure relates to the use of mitragynine (MG) and naltrexone (NTX) in combination in the treatment of substance use disorders. The use of MG and NTX as a combination therapy for substance use disorders such as AUD is new and contrary to expectation. In fact, mu antagonists like naltrexone have been shown to block MG's analgesic effects. That combining MG with NTX and finding an enhanced effect in decreasing alcohol self-administration is unexpected.

MG is the primary alkaloid among more than 40 unique alkaloids found in leaves of the tree Mitrgyna speciose (kratom) that is indigenous to Southeast Asia. Traditionally, the leaves from this tree are used for pain relief or as an aid for manual labor because of its mild psychostimulant effects. Individuals in the United States who consume kratom use it to reduce pain, anxiety, alleviate depression, and to reduce opioid withdrawal according to survey data. And, those who used kratom to reduce opioid withdrawal also showed decreased alcohol intake. The few preclinical studies that exist demonstrate that MG or kratom extracts attenuates alcohol withdrawal, alcohol-seeking, alcohol consumption in mice and alcohol withdrawal in rats. Only one of these studies included females. Further, whether MG alters alcohol reinforcement in an operant alcohol self-administration procedure was unknown until the studies contained in this application were conducted.

The use of kratom to self-medicate pain or opioid withdrawal in humans is thought to reflect its action as a partial mu opioid agonist or full agonist. Evidence however suggests that it may act as a mu antagonist or have no intrinsic activity in and of itself but is indeed a pro-drug that is metabolized into the potent mu agonist 7-hydroxymitragynine (7-HMG). Nevertheless, if MG does act as a mu opioid antagonist, then it should reduce alcohol self-administration consistent with the abundant research on NTX. Alternatively, if MG has mu agonist-like effects, it might increase alcohol self-administration similar to findings from some studies with morphine and other opioids. Other studies however report that opioids reduce the behavioral effects of alcohol. Given that the anti-nociception effects of MG are blocked by the mu opioid antagonist naloxone, it is anticipated that NTX will attenuate any potential agonist-like effects of MG on operant alcohol self-administration.

Studies were performed to test whether MG attenuates operant alcohol self-administration behaviors in a manner similar to NTX in female rats. Further tests were conducted to determine whether NTX alters MG-induced effects. The impact of the highest dose of MG and NTX alone and the combination were tested to determine the potential impact on locomotor activity. Immediate early gene expression was examined in various brain regions using cFos expression as an indirect marker of neuronal activity in response to administration of MG, NTX, and their combination.

Unexpectedly, the combination of MG and NTX was more impactful than either alone and engendered no adverse effects on locomotor activity. That NTX does not block MG-induced decreases of operant alcohol self-administration is surprising and contrary to current understandings. Compared to naloxone, NTX is a longer-acting mu opioid antagonist that would presumably block MG's analgesic effects. Combining MG and NTX therefore results in a unique, unexpected, and beneficial treatment that decreases alcohol self-administration to a greater extent than either alone. NTX is indicated for the treatment of AUD and OUD however its effectiveness is poor due to numerous factors. Therefore, treatment efficacy of NTX may be increased when combined with MG than when administered alone. In addition, other treatment options presently on the market (such as disulfiram and acamprosate) have shown poor efficacy and compliance, highlighting the need for better pharmacotherapies for AUD.

Importantly, individuals with AUD often have multiple co-occurring psychiatric disorders. Evidence from surveys conducted in the USA indicate that individuals consume kratom to alleviate anxiety and depression two disorders commonly associated with AUD. Animal data also supports the notion that MG possesses anxiolytic and antidepressant effects. Thus, the present drug combination is the only treatment for AUD that might address other underlying comorbidities linked to AUD.

### BRIEF DESCRIPTION OF THE DRAWINGS

FIG. 1 shows the effects of mitragynine (0; 0.3-3.0 mg/kg; IP) on operant alcohol (10%) self-administration in female Sprague Dawley rats (N=10) during 60-min sessions conducted under an FR2 schedule of reinforcement, including (A) active (open bars) and inactive (closed bars) lever presses; (B) numbers of head entries; (C) numbers of reinforcers earned; (D) estimated total alcohol consumed.

FIG. 2 shows effects of naltrexone (0; 0.3-3.0 mg/kg; IP) on operant alcohol (10%) self-administration in female Sprague Dawley rats (N=10) during 60-min sessions conducted under an FR2 schedule of reinforcement, including (A) active (open bars) and inactive (closed bars) lever presses; (B) numbers of head entries; (C) numbers of reinforcers earned; (D) estimated total alcohol consumed.

FIG. 3 shows effects of the combination of equal doses of mitragynine and naltrexone (0; 0.3-3.0 mg/kg; IP) on operant alcohol (10%) self-administration in female Sprague Dawley rats (N=10) during 60-min sessions conducted under an FR2 schedule of reinforcement, including A) active (open bars) and inactive (closed bars) lever presses; B) numbers of head entries; C) numbers of reinforcers earned; D) estimated total alcohol delivered.

FIG. 4 shows direct comparisons of the effects of mitragynine alone (circles), naltrexone alone (squares), and the combination (triangles) on operant alcohol (10%) self-administration in female Sprague Dawley rats (N=10) during 60-min sessions conducted under an FR2 schedule of reinforcement, including (A) active lever presses; (B) numbers of head entries; (C) numbers of reinforcers earned; (D) estimated total alcohol delivered.

FIG. 5 shows effects of vehicle (control; open circles), mitragynine (closed squares), naltrexone (closed upward triangles), and the combination (closed downward triangles) (3 mg/kg) on distance traveled (cm) over 5-min time blocks in (A) an open field test and (B) over the entire 30-min open field test session in female Sprague Dawley rats (N=4/group).

FIG. 6 shows effects of vehicle (control), mitragynine, naltrexone, and the combination (3 mg/kg) on cFos activation in various brain areas in female Sprague Dawley rats (N=4/group) examined 90-min after the open field test, including (A) a representative slice of prelimbic (PL) and infralimbic (IL) cortices showing cFos expression, and other brain areas surveyed including (B) the nucleus accumbens (NAC), (C) dorsal striatum (Str), (D) basolateral amygdala (BLA), (E) central nucleus of the amygdala (CeA), (F) infralimbic cortex (IL), (G) prelimbic cortex (PL), (H) cingulate cortex area 1 (Cg1), (I) cingulate cortex area 2 (Cg2).

### DETAILED DESCRIPTION OF PREFERRED EMBODIMENTS

The present disclosure relates to a therapeutic drug combination including mitragynine (MG) and naltrexone (NTX). The drug combination may be useful for the treatment of substance use disorders including alcohol use disorder (AUD) and opioid use disorder (OUD).

Preferred embodiments relate to a therapeutic drug combination comprising MG and NTX. Further preferred embodiment relate to a therapeutic drug combination consisting essentially of MG and NTX and lacking any other active ingredients. Additional preferred embodiments relate to methods for treatment of substance use disorders comprising the step of administering a therapeutic drug combination comprising MG and NTX, or consisting essentially of MG and NTX, without other active ingredients. The substance use disorder may be AUD or OUD. In preferred embodiments, the therapeutic drug combination comprises equal concentrations of MG and NTX. In additional preferred embodiments, the therapeutic drug combination comprises concentrations of MG and NTX in a ratio of MG:NTX of between 1:5 and 1:10.

Additional preferred embodiments relate to a pharmaceutical composition for administration to a subject including a therapeutically effective amount of a therapeutic drug combination comprising MG and NTX and a pharmaceutically acceptable excipient, adjuvant, carrier, buffer or stabilizer. A “therapeutically effective amount” is to be understood as an amount of an exemplary therapeutic drug combination comprising MG and NTX that is sufficient to show a positive biological effect on a substance use disorder being treated. The actual amount, rate and time-course of administration will depend on the nature and severity of the disorder being treated. Prescription of treatment is within the responsibility of general practitioners and other medical doctors. The pharmaceutically acceptable excipient, adjuvant, carrier, buffer or stabilizer should be non-toxic and should not interfere with the efficacy of the active ingredient. The precise nature of the carrier or other material will depend on the route of administration, which may be oral, or by injection, such as cutaneous, subcutaneous, or intravenous injection, or by dry powder inhaler. In preferred embodiments, the route of administration is oral.

In additional preferred embodiments, the pharmaceutical composition comprises appropriate dosages of MG and NTX, based on weight (in kg) of the subject to whom the composition is administered. Dosages used in the examples below were suitable for animals. Suitable dosages for humans can be calculated based on knowledge of those in the art (Reagan-Shaw S, Nihal M, Ahmad N., FASEB J. 2008 March; 22 (3): 659-61). Body surface area is taken into consideration between species. Based on the dosage used in the examples (0.3 mg/kg, 1.0 mg/kg, and 3.0 mg/kg), exemplary dosages for humans include 3.4 mg/70 kg human, 11.35 mg/70 kg human, and 33.6 mg/70 kg human. This is about 0.049 mg/kg to about 0.48 mg/kg in humans that weigh 70 kg. In larger humans about 80-90 kg in weight the higher end of the dosage range is preferably 39-50 mg/day. The typical indicated dose of NTX alone for use in treatment of AUD is 50 mg/day.

Pharmaceutical compositions for oral administration may be in tablet, capsule, powder or liquid form. A tablet may comprise a solid carrier or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. A capsule may comprise a solid carrier such as gelatin. For intravenous, cutaneous or subcutaneous injection, the active ingredient will be in the form of a parenterally acceptable aqueous solution which is pyrogen-free and has a suitable pH, isotonicity and stability. Those of relevant skill in the art are well able to prepare suitable solutions using, for example, isotonic vehicles such as sodium chloride solution, Ringer's solution, or lactated Ringer's solution. Preservatives, stabilizers, buffers, antioxidants and/or other additives may be included as required.

In another aspect, there is provided the use in the manufacture of a medicament a therapeutically effective amount of a therapeutic drug combination comprising MG and NTX as defined above for administration to a subject.

The term “therapeutically effective amount” means a nontoxic but sufficient amount of the drug to provide the desired therapeutic effect. The amount that is “effective” will vary from subject to subject, depending on the age and general condition of the individual, the particular concentration and composition being administered, and the like. Thus, it is not always possible to specify an exact effective amount. However, an appropriate effective amount in any individual case may be determined by one of ordinary skill in the art using routine experimentation. Furthermore, the effective amount is the concentration that is within a range sufficient to permit ready application of the formulation so as to deliver an amount of the drug that is within a therapeutically effective range.

Further aspects of the present invention will become apparent from the following description given by way of example only.

### EXAMPLES

### Materials and Methods

All procedures were approved by the University of Houston Institutional Animal Care and Use Committee in accordance with the National Institutes of Health Guidelines.

Animals. A total of 10 female Sprague Dawley rats (Charles River, Wilmington, MA) were used for the alcohol self-administration studies. Separate groups of female rats (N=16) were used to assess the impact of vehicle (control, N=4), MG (N=4, 3.0 mg/kg), NTX (N=4, 3 mg/kg) and MG+NTX (N=4, 3.0 mg/kg/3.0 mg/kg) on locomotor activity in open field chambers. These same rats were used in the cFos immunohistochemistry study. Rats were initially housed 3-5 to a cage in polypropylene cages housed in circular towers (Animal Care Systems, Inc, Centennial, CO) located within a temperature- and humidity-controlled vivarium that was maintained on a 12:12 light/dark cycle (lights on at 6 AM). Rats weighed about 250-350 gm at the start of the experiment and were at least 100 days old. Food and water were available ad libitum throughout the study. To facilitate alcohol self-administration rats were exposed to alcohol in vapor chambers (La Jolla Alcohol Research, La Jolla, CA) for 6-weeks prior to operant training using a chronic intermittent alcohol exposure (i.e., alcohol vapors were on for 14-h and off for 10-h, 5 days per week).

Drugs. Alcohol (ethyl alcohol, 190 Proof, USP grade, Koptec, King of Prussia, PA) was diluted to 10% (with RO water, w/v) and made available for consumption via standard operant chambers (described below). MG (Cayman Chemical, Ann Arbor, MI) was prepared in 20% Tween 80 (Sigma Aldrich, St. Louis, MO) in 80% sterile saline (0.9% NaCl). NTX was purchased from Sigma Aldrich and prepared in sterile saline. Doses of MG and NTX were chosen based on previously published studies. Each dose of test drug was administered via intraperitoneal (IP) injection.

Operant self-administration training and testing. Training sessions (60 min) began with the illumination of the house light and, initially, two non-contingent dipper presentations (primes) for 10 sec. The dipper access light was illuminated for the entire length of the dipper presentation time. Dipper presentation times were gradually reduced (10>5>3 seconds) over subsequent weeks of training, based on each animal's performance, until the dipper presentation was 3 seconds in duration. Three cue lights were illuminated above both the active and inactive levers. When the rat pressed the active lever, the house light would turn off, dipper would protrude, and the access area light and the triple cue light above both of the levers went off. Presses on the inactive lever had no consequences. Once a rat emitted 20 or more active lever presses with 20% variability or less in response levels over 2 consecutive days, the ratio requirement was raised to fixed-ratio 2 (FR2), which was the schedule used for the rest of the training. Stable response levels under the FR2 schedule (≤20% variability over 2 consecutive days) under 3 second dipper presentation time were required for the animal to move into the testing phase. Test sessions (60 min) were conducted once a rat met stable lever pressing criteria. Each dose of test drug and vehicle was administered (IP) 30 minutes prior to the test session in a randomized order across rats with at least 4-7 days intervening between dose administrations.

Responses obtained from the test sessions included two measures of appetitive responding (numbers of active lever presses and head entries) and two measures of consummatory responding (numbers of reinforcers earned and estimated amount of alcohol consumed in g/kg). Estimates of the amount of alcohol delivered were derived by multiplying the number of reinforcers earned by the amount of alcohol (g) per delivery (0.1 mL) of the 10% solution and divided by body weight. Numbers of active vs. inactive lever presses were compared and analyzed to demonstrate that rats had acquired the lever discrimination.

Open Field Test-Locomotor Activity. To assess potential non-specific effects on locomotor activity, four separate groups of rats were assigned to a specific drug condition: saline (control), MG, NTX, and the combination. Drugs were administered 30 minutes before rats were placed into the open field chambers and distance traveled (cm) was tabulated in 5-min blocks across the 30-minute test.

Immunohistochemistry: c-Fos. Ninety minutes after the open field test, the rats were anaesthetized with (ketamine 91 mg/kg; xylazine 9.1 mg/kg) and transcardially perfused with 0.9% saline followed by 4% paraformaldehyde in 0.1 M phosphate-buffered saline (PBS) (pH 7.4). Brains were removed, immersed (4° C.) in the above fixative for 2 h and then kept in 30% sucrose in 0.1 M PBS until soaked. The 40-μm thick brain slices were obtained; having as reference the following, AP coordinates: bregma PL/IL+3.20, NAC+1.20, Cg1/Cg2+1.00, Dorsal striatum+0.7 and BLA/CeA-2.56 mm). The slices were collected in 0.1 M PBS and subsequently processed free-floating according to the avidine-biotine procedure, using the VECTASTAIN® Elite® ABC Universal PLUS Kit, Peroxidase (Horse Anti-Mouse/Rabbit IgG) PK-8200 (Vector, USA, Ref. PK 8200). All reactions were carried out under agitation, at room temperature. The slices were first incubated with BLOXALL endogenous enzyme blocking solution for 10 min, washed four times with 0.1 M PBS (5 min each) and then incubated overnight with the primary Fos polyclonal antibody (Santa Cruz, USA, SC-52) at a concentration of 2/2000 in 0.1 M PBS. Slices were again washed three times (5 min each) with 0.1 M PBS and incubated for 1 h with biotinylated horse anti-mouse/rabbit IgG secondary antibody. After another series of three 5-min washings in 0.1 M PBS, they were incubated for 1 h with VECTASTAIN Elite ABC reagent and then washed for 5 minutes in PBS. The slices were finally allowed to remain in mix ImmPACT DAB EqV solution in 1:1 ratio for required volume and incubated on the section until appropriate stain intensity developed. The slices were then rinsed with water then mounted with DPX.

Quantification of fos-positive cells. Sections were mounted on gelatin-coated slides, dehydrated and cover slipped for observation and cell counting under bright-field microscopy. The nomenclature and nuclear boundaries utilized were based on the atlas of Paxinos G, Watson C (2013) The Rat Brain in Stereotaxic Coordinates. Academic Press, San Diego. Neuronal nuclei expressing levels of DAB reaction product above tissue background were automatically counted by a computerized image analysis system (Image J). Briefly, mounted sections of the tissue were observed using a light microscope (Nikon Eclipse Ti2 Microscope with NIS-Elements AR software, Nikon Instrumentws Inc. USA) equipped with a video camera module (Hamatsu Photonics C2400). Counting of Fos-positive cells was performed at a magnification ×10, in one field per area encompassing the entire brain region included in quantification. An area of the same shape and size per brain region was used for each rat. The same light and threshold conditions were employed for all sections. In order to ensure accuracy of measurement and avoid variations among same areas in different subjects, the background of every area was measured and digitally subtracted from the area under examination. Accordingly, the threshold conditions were set for each area and maintained for all subjects. All brain regions were bilaterally counted in various sections for each rat depending on the size of the structure. After that, counts for each region were averaged over the sections. Nuclei were counted individually and expressed as number of Fos-positive nuclei per 0.1 μm².

Statistical Analysis. The self-administration data were analyzed with ANOVAs to compare the two measures of appetitive responding (numbers of active lever presses and head entries) and the two measures of consummatory responding (numbers of reinforcers earned and estimated total alcohol intake in g/kg). Separate one-way ANOVAs were conducted for each drug (MG and NTX) and for the combination with Dose (0, 0.3, 1.0, and 3.0 mg/kg) as the repeated measure. All three Drug conditions (MG, NTX, and MG+NTX) were compared in 3×4 ANOVAs with Dose as the repeated measure for each of the four measures. In addition, the factor of Lever (active vs. inactive) was included in the ANOVAs to assess lever discrimination for each Drug and for the combined drug condition analysis.

Distance traveled over time was analyzed using a 4×6 repeated measures ANOVA with time (six 5-min blocks) as the repeating measure. Another ANOVA was conducted that included all three Drug conditions (MG, NTX, and MG+NTX). Total distance traveled (cm) in the open field study and cFos activation data were analyzed using One-Way ANOVA to test the Group factor of Drug (vehicle, MG, NTX, and MG+NTX).

Significant main effects were followed by Tukey's multiple comparisons test. Comparisons with p-values less than 0.05 were considered significant and those less than 0.10 were considered a trend towards significance.

### Results

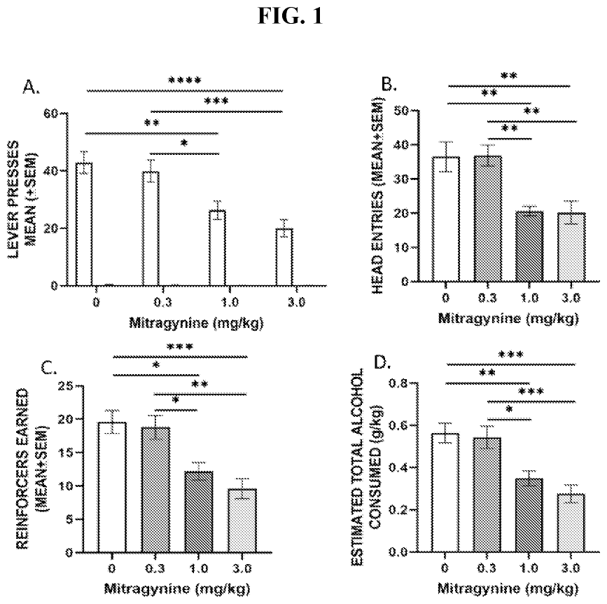

Alcohol self-administration. All 10 female rats acquired the operant and were included in the study. FIG. 1 shows the effects of mitragynine (0; 0.3-3.0 mg/kg; IP) on operant alcohol (10%) self-administration in female Sprague Dawley rats (N=10) during 60-min sessions conducted under an FR2 schedule of reinforcement, including A) active (open bars) and inactive (closed bars) lever presses; B) numbers of head entries; C) numbers of reinforcers earned; D) estimated total alcohol consumed. Data are presented as Mean±SEM. All measures were significantly decreased by mitragynine. *P<0.05, **P<0.01, ***P<0.001, ****P<0.0001.

The effects of vehicle and three doses of MG on active vs. inactive lever presses are shown in FIG. 1A. ANOVA revealed significant main effects for Lever (F_{(1, 72)}=342, P<0.0001), Dose (F_{(3, 72)}=10.05, P<0.0001), and a significant interaction (F_{(3, 72)}=9.74, P<0.0001). Rats discriminated between active and inactive lever as seen in FIG. 1A and supported by the significant main effect of Lever. That is, numbers of active lever presses were much greater than the very minimal numbers of inactive lever presses emitted. Post-hoc multiple comparisons between drug doses showed significant differences in active lever presses between vehicle and 1.0 (P<0.01) and 3.0 mg/kg (P<0.0001) MG. Further, active lever presses also differed significantly between 0.3 and 1.0 (P<0.05) and 3.0 mg/kg (P<0.001) MG. FIG. 1B shows the effects of MG on number of head entries. ANOVA indicated a significant main effect for Dose (F_{(3, 36)}=8.44, P<0.001). Post-hoc multiple comparisons test revealed significant differences between vehicle and 1.0 (P<0.01) and 3.0 mg/kg (P<0.01) as well as between 0.3 and 1.0 (P<0.01) and 3.0 mg/kg (P<0.01).

FIG. 1C presents the number of reinforcers earned following vehicle and various doses of MG. ANOVA revealed a significant main effect for Dose (F_{(3, 36)}=9.61, P<0.0001). Post-hoc multiple comparisons test revealed significant differences between vehicle compared to 1.0 (P<0.05) and 3.0 mg/kg (P<0.0001) and between 0.3 and 1.0 (P<0.05) and 3.0 mg/kg MG on reinforcers earned. Estimated total alcohol intake (g/kg) following vehicle and the various doses of MG are presented in FIG. 1D. ANOVA revealed a significant main effect for Dose (F_{(3, 36)}=10.13, P<0.0001). Post-hoc analysis indicated significant differences between vehicle and 1.0 (P<0.01) and 3.0 mg/kg (P<0.001). Additionally, significant differences were also found between 0.3 and 1.0 (P<0.05) and 3.0 mg/kg (P<0.001).

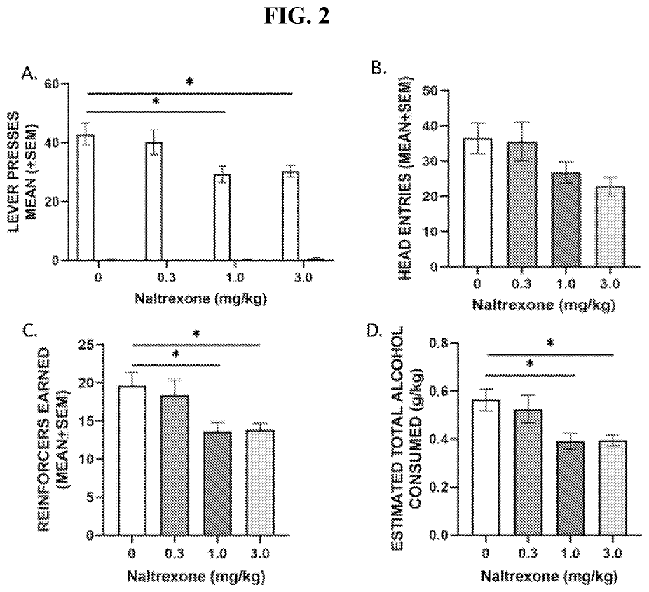

FIG. 2 shows effects of naltrexone (0; 0.3-3.0 mg/kg; IP) on operant alcohol (10%) self-administration in female Sprague Dawley rats (N=10) during 60-min sessions conducted under an FR2 schedule of reinforcement, including A) active (open bars) and inactive (closed bars) lever presses; B) numbers of head entries; C) numbers of reinforcers earned; D) estimated total alcohol consumed. Data are presented as Mean±SEM. All measures were significantly decreased by naltrexone except for head entries that showed a trend towards significance. +P<0.10 (trend); *P<0.05, **P<0.01, ***P<0.001, ****P<0.0001.

The effects of vehicle and three doses of NTX on active and inactive lever presses are shown in FIG. 2A. ANOVA revealed significant main effects for Lever (F_{(1, 72)}=464.4, P<0.0001), Dose (F_{(3.72)}=4.286, P<0.01) and a significant interaction (F_{(3.72)}=4.519, P<0.01). Rats discriminated between active and inactive lever as seen in FIG. 2A and supported by the significant main effect of Lever. Post-hoc multiple comparisons between drug doses showed significant differences in active lever presses between vehicle and 1.0 (P<0.05) and 3.0 mg/kg (P<0.05) NTX. FIG. 2B shows the effects of NTX on number of head entries. ANOVA indicated a trend towards a main effect for Dose (F_{(3, 36)}=2.712, P=0.06).

FIG. 2C presents the number of reinforcers earned following vehicle and various doses of NTX. ANOVA revealed a significant main effect for Dose (F_{(3, 36)}=4.122, P<0.05). Post-hoc multiple comparisons tests revealed significant differences between vehicle compared to 1.0 (P<0.05) and 3.0 mg/kg (P<0.05). The effects of NTX on estimated total alcohol intake is displayed in FIG. 2D. ANOVA indicated a significant main effect for Dose (F_{(3, 36)}=4.41, P<0.01). Post-hoc analysis showed significant differences between vehicle and 1.0 (P<0.05) and 3.0 mg/kg (P<0.05) NTX.

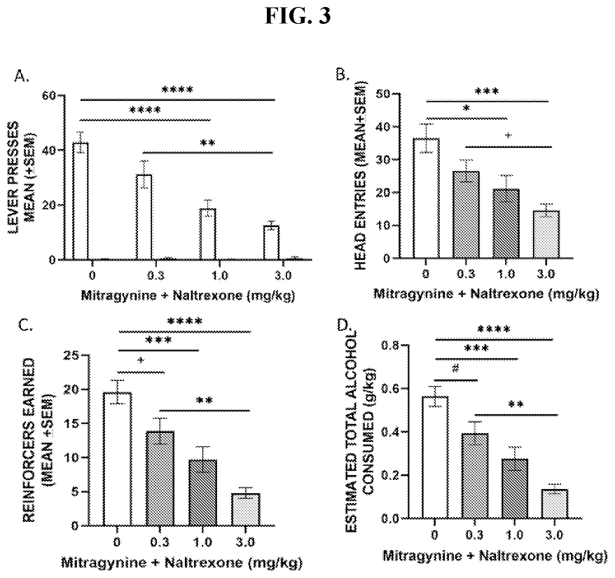

FIG. 3 shows effects of the combination of equal doses of mitragynine and naltrexone (0; 0.3-3.0 mg/kg; IP) on operant alcohol (10%) self-administration in female Sprague Dawley rats (N=10) during 60-min sessions conducted under an FR2 schedule of reinforcement, including A) active (open bars) and inactive (closed bars) lever presses; B) numbers of head entries; C) numbers of reinforcers earned; D) estimated total alcohol delivered. Data are presented as Mean±SEM. All measures were significantly decreased by the combination. *P<0.05, **P<0.01, ***P<0.001, ****P<0.0001.

The effects of vehicle and the combination of MG+NTX on active and inactive lever presses are shown in FIG. 3A. ANOVA revealed significant main effects for Lever (F_{(1, 72)}=217.1, P<0.0001), Dose (F_{(3, 72)}=14.39, P<0.0001) and a significant interaction (F_{(3, 72)}=14.57, P<0.0001). Rats discriminated between active and inactive lever as seen in FIG. 3A and supported by the significant main effect of Lever. Post-hoc multiple comparisons between drug doses showed significant differences in active lever presses between vehicle and 1.0 (P<0.0001) and 3.0 mg/kg (P<0.0001) MG+NTX. Further, active lever presses also differed significantly between 0.3 and 3.0 mg/kg (P<0.0001) MG+NTX and showed a trend towards a difference between 0.03 and 1.0 mg/kg (P=0.06). FIG. 3B shows the effects of MG+NTX on number of head entries. ANOVA indicated a significant main effect for Dose (F_{(3, 36)}=6.88, P<0.001). Post-hoc multiple comparisons test revealed significant differences between vehicle and 1.0 (P<0.05) and 3.0 mg/kg (P<0.001) as well as a trend for a significant difference between 0.3 and 3.0 mg/kg (P<0.09).

FIG. 3C presents the number of reinforcers earned following vehicle and various doses of MG+NTX. ANOVA revealed a significant main effect for Dose (F_{(3, 36)}=14.64, P<0.0001). Post-hoc multiple comparisons test revealed significant differences between vehicle compared to 1.0 (P<0.001) and 3.0 mg/kg (P<0.0001) and between 0.3 and 3.0 mg/kg MG+NTX on reinforcers earned. Analysis also revealed a trend towards a significant difference between vehicle and 0.3 mg/kg (P=0.08) of the combination. The effects of MG+NTX on estimated total alcohol consumed is presented in FIG. 3D. ANOVA revealed a significant main effect for Dose (F_{(3, 36)}=15.63, P<0.0001). Post-hoc analysis showed a trend towards a significant difference between vehicle and 0.3 mg/kg (P=0.057) and significant differences between vehicle and 1.0 (P<0.001) and 3.0 mg/kg (P<0.0001) MG+NTX. In addition, there was a significant difference between 0.3 and 3.0 mg/kg (P<0.01) MG+NTX.

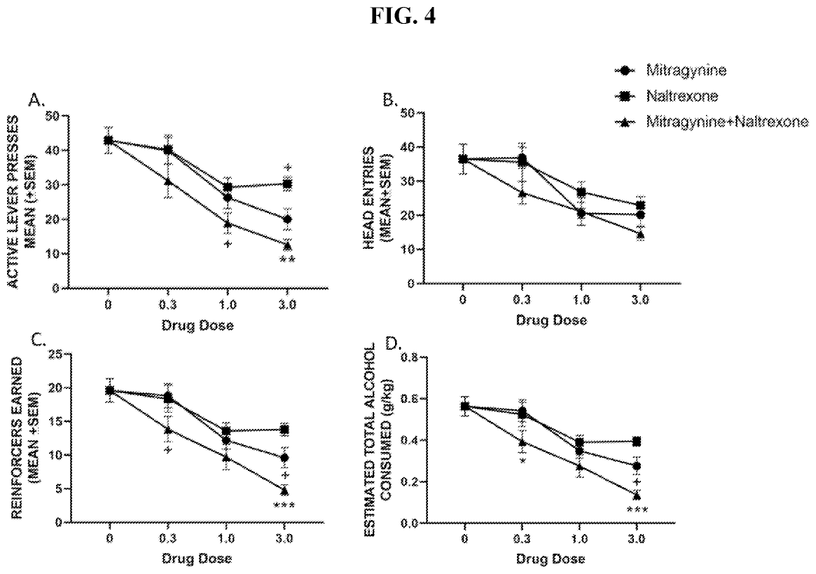

Direct between group comparisons across drug. Direct between group comparisons across dose for MG, NTX and MG+NTX (MG=mitragynine, NTX=naltrexone) are presented in FIG. 4. FIG. 4 shows direct comparisons of the effects of mitragynine alone (circles), naltrexone alone (squares), and the combination (triangles) on operant alcohol (10%) self-administration in female Sprague Dawley rats (N=10) during 60-min sessions conducted under an FR2 schedule of reinforcement, including A) active lever presses; B) numbers of head entries; C) numbers of reinforcers earned; D) estimated total alcohol delivered. Data are presented as Mean±SEM. +P=0.1, **P<0.01, ***P<0.001. Analysis was performed on active lever presses (4A), head entries (4B), reinforcers earned (4C) and estimated total alcohol consumed (4D). For active lever presses, ANOVA revealed a significant main effect for Drug (F_{(2, 108)}=7.52, P<0.001), Dose (F_{(3, 108)}=27.06, P<0001) but no interaction (F_{(6, 108)}=1.303, P=0.26). Post-hoc analysis showed a significant difference between NTX and MG+NTX (P<0.01) at the 3.0 mg/kg dose. Trends towards significance were seen between NTX and MG+NTX at 1.0 mg/kg and between MG and NTX at the 3.0 mg/kg dose (P=0.08). For head entries, ANOVA revealed a trend towards a main effect for Drug (F_{(2, 108)}=2.60, P=0.08), but a significant main effect for Dose (F_{(3, 108)}=15.33, P<0.0001) and no interaction (F_{(6, 108)}=0.68, P=0.66).

For reinforcers earned, ANOVA revealed a significant main effect for Drug (F_{(2, 108)}=7.96, P<0.001), Dose (F_{(3, 108)}=26.06, P<0.0001) but no interaction (F_{(6, 108)}=1.55, P=0.16). Post-hoc analysis showed a significant difference between NTX and MG+NTX (P<0.001) at the 3.0 mg/kg dose. Trends towards significance were seen between MG and MG+NTX (P=0.07) at 0.3 mg/kg and between MG and MG+NTX at the 3.0 mg/kg dose (P=0.08). Direct comparisons between groups across dose for estimated total alcohol consumed are presented in FIG. 4D. ANOVA revealed a significant main effect for Drug (F_{(2, 108)}=8.62, P<0.001), Dose (F_{(3, 108)}, 27.72, P<0.0001) but no interaction (F_{(6, 108)}=1.62, P=0.134). Post-hoc analysis showed a significant difference between MG and MG+NTX at the 0.3 mg/kg (P<0.05) dose and between NTX and MG+NTX (P<0.001) at the highest dose (3.0 mg/kg). Additionally, there was a trend towards a significant difference between MG and MG+NTX at the 3.0 mg/kg (P=0.07).

The degree of significance was also compared for the Dose factor across drugs on all alcohol self-administration measures including appetitive (numbers of active lever presses and head entries) and consummatory (numbers of reinforcers earned and estimate alcohol amount delivered) responses. As seen in Table 1 below, all effects were significant except for numbers of head entries after NTX administration that showed a trend towards significance. The greatest significance was seen for the combination of MG+NTX vs either drug alone except for the number of head entries in which MG had the greatest effect. Across all four measures, the drug condition with the least significant effects was NTX.

Table 1 shows comparisons of F values for Dose effects in analyses of MG and NTX alone and the combination on appetitive (active lever presses and head entries) and consummatory (reinforcers earned and estimated alcohol intake) measures. All Dose effects were significant except for number of head entries with NTX that showed a trend for significance (p<0.06) and is shown in bold type.

TABLE 1

| Response | | | | MG + |
|---|---|---|---|---|
| Type | Measure | MG | NTX | NTX |
| Appetitive | # active lever presses | 10.05 | 4.29 | 14.39 |
| | # head entries | 8.44 | 2.71 | 6.88 |
| Consummatory | # reinforcers earned | 9.61 | 4.12 | 14.64 |
| | Estimated amount | 10.13 | 4.41 | 27.72 |

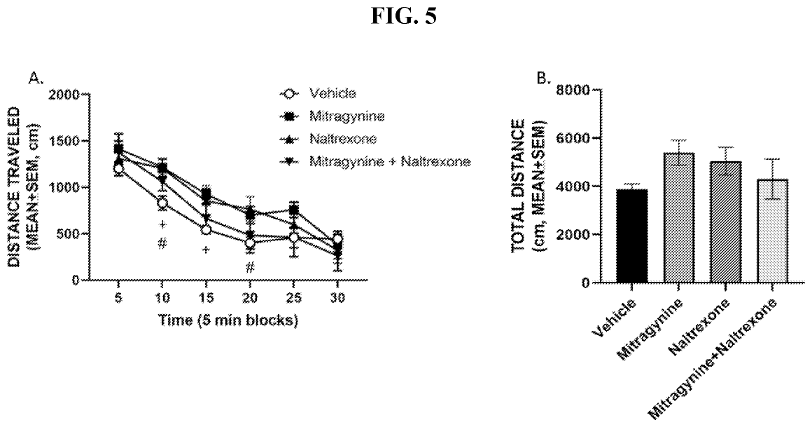

Open Field Test. FIG. 5 shows effects of vehicle (control; open circles), mitragynine (closed squares), naltrexone (closed upward triangles), and the combination (closed downward triangles) (3 mg/kg) on distance traveled (cm) over 5-min time blocks in (A) an open field test and (B) over the entire 30-min open field test session in female Sprague Dawley rats (N=4/group). Distance traveled decreased over time however mitragynine and naltrexone tend to increase locomotor activity (+Control vs mitragynine, P=0.08; #Control vs. naltrexone, P=0.09). Total distance traveled did not differ between groups. Distance traveled (cm) over the six 5-min time blocks and total distance across the 30-min session following vehicle, MG (3.0 mg/kg), NTX (3.0 mg/kg), and MG (3 mg/kg)+NTX (3 mg/kg) are presented in FIGS. 5A and B respectively. Analysis of distance traveled over time following dosing revealed a significant main effect for Drug (F_{(3, 72)}=5.38, P>0.01) and Time (F_{(5, 72)}=35.69, P<0.0001) but no interaction (F_{(15, 72)}=0.655, P=0.81. Post-hoc analysis showed trends towards significant differences between vehicle and MG (P=0.08) and NTX (P=0.09) at 10 minutes, vehicle and MG (P=0.08) at 15 minutes and vehicle and NTX (P=0.09) at 20 minutes. For total distance traveled across Drug groups (FIG. 4B), ANOVA revealed no significant main effect (F_{(3, 24)}=0.333, P=0.80).

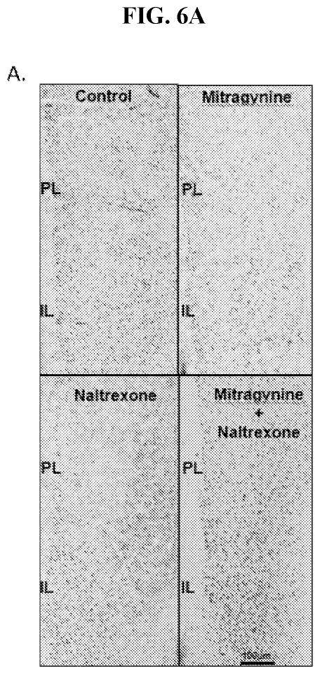

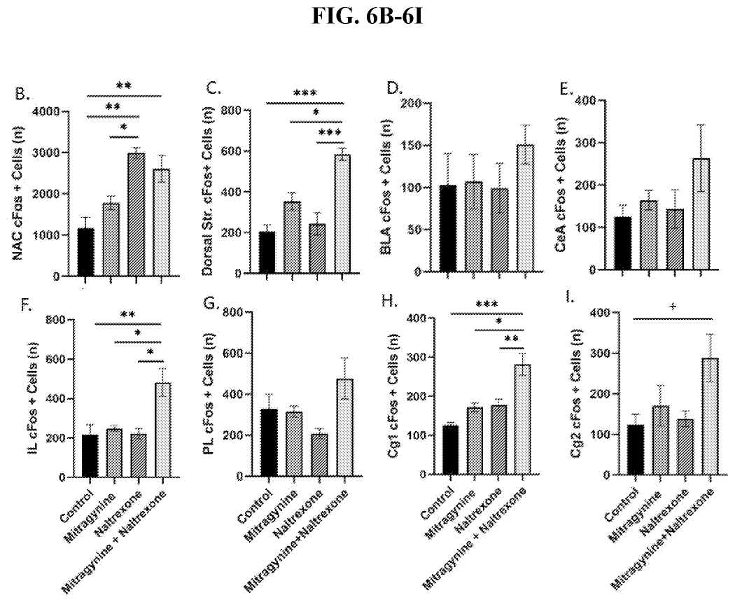

Immunohistochemistry: cFos activation. Results from immunohistochemistry staining for cFos expression in various brain areas are presented in FIG. 6. FIG. 6 shows effects of vehicle (control), mitragynine, naltrexone, and the combination (3 mg/kg) on cFos activation in various brain areas in female Sprague Dawley rats (N=4/group) examined 90-min after the open field test. Panel A is a representative slice of prelimbic (PL) and infralimbic (IL) cortices showing cFos expression. Other brain areas surveyed include the nucleus accumbens (NAC; B) dorsal striatum (Str; C), basolateral amygdala (BLA; D), central nucleus of the amygdala (CeA; E), infralimbic cortex (IL; F), prelimbic cortex (PL; G), cingulate cortex area 1 (Cg1; H), cingulate cortex area 2 (Cg2; I). +P<0.1, *P<0.05, **P<0.01, ***P<0.001. FIG. 6A is a representative sample from four rat's pre-limbic (PL) and infralimbic (IL) cortex after vehicle (control), MG (3 mg/kg), NTX (3 mg/kg) or MG (3 mg/kg)+NTX (3 mg/kg) administration. FIG. 6B presents numbers of cFos positive neurons in the NAC. Analysis of the number of cFos activated cells in the NAC revealed a significant main effect for Drug (F (3, 11)=10.67, P=0.001). Post-hoc multiple comparison tests revealed a significant difference between control and NTX (P=0.002) and MG+NTX (P=0.008), in addition to MG and NTX (P<0.05). FIG. 6C presents numbers of cFos positive neurons in the dorsal striatum (Dorsal Str). Analysis of the number of cFos activated cells in the Dorsal Str revealed a significant main effect for Drug (F_{(3, 12)}=17.07, P=0.0001). Post-hoc multiple comparison tests revealed a significant difference between vehicle (control) and MG+NTX (P<0.001) and MG (P<0.05) and NTX (P<0.001) compared to MG+NTX. FIG. 6F presents numbers of cFos positive neurons in the IL cortex. Analysis of the number of cFos activated cells in the IL cortex revealed a significant main effect for Drug (F_{(3, 11)}=7.90, P=0.004). Post-hoc multiple comparison tests revealed significant differences between vehicle (control) and MG+NTX (P<0.05) and MG (P<0.05) and NTX (P<0.01) compared to MG+NTX. FIG. 6H presents numbers of cFos positive neurons in the cingulate cortex 1 (Cg1). Analysis of the number of cFos activated cells in the Cg1 cortex revealed a significant main effect for Drug (F_{(3, 11)}=14.03, P=0.0004). Post-hoc multiple comparison tests revealed a significant difference between vehicle (control) and MG+NTX (P=0.0004) and MG (P<0.05) and NTX (P<0.01) compared to MG+NTX. FIG. 6I presents numbers of cFos positive neurons in the cingulate cortex 2 (Cg2). Analysis of the number of cFos activated cells in the Cg2 cortex revealed a significant main effect for Drug (F_{(3, 11)}=3.60, P=0.049). Post-hoc multiple comparison test revealed a trend towards a significant difference between vehicle (control) and MG+NTX (P=0.07). No other significant differences were identified between drug treatment groups among the other brain areas surveyed.

### DISCUSSION

This study assessed the impact of MG alone and in combination with NTX on oral operant alcohol self-administration in female rats. MG and NTX alone reduced various measures of alcohol self-administration with MG showing greater efficacy. Significant decreases in self-administration measures were not due to non-specific effects as evidenced by the lack of finding that either drug depressed locomotor activity. Unexpectedly, the combination of MG+NTX reduced alcohol self-administration and amount consumed to a greater extent than either drug alone. Similarly, the combination of MG+NTX induced greater numbers of cFos expressing neurons in brain areas known to mediate alcohol reinforcement compared to either drug administered alone. Overall, these data demonstrate that MG and NTX appear to act in coordination to reduce oral operant alcohol self-administration in rats. Evidence also suggests that the ability of MG to decrease alcohol-related behaviors is likely mediated by non-opioid mechanisms.

Effects of NTX alone on alcohol self-administration. Previous work showing NTX decreases various measures of oral operant alcohol self-administration in female Sprague Dawley rats were replicated even though there were minor differences in the outcomes. NTX pre-treatment decreased active lever presses and reinforcers earned at the two highest doses tested (1.0 and 3.0 mg/kg, FIGS. 2A & 2C) whereas there was a trend towards a significant decrease in head entries. In our previous study, NTX decreased active lever presses, reinforcers earned but not head entries, at the highest NTX dose (10 mg/kg) in female Sprague Dawley rats. Variances between studies regarding the most efficacious dose of NTX to decrease alcohol self-administration is likely due to differences in experimental procedures employed. That is, a progressive ratio schedule of reinforcement was used in the previous study whereas in this study a fixed ratio (2) schedule was used. In addition, and consistent with the results herein, numerous studies using mostly male rats and utilizing a broad spectrum of experimental procedures show that NTX consistently reduces alcohol self-administration and consumption.

The exact mechanism(s) responsible for the ability of NTX to decrease alcohol self-administration is somewhat delineated in studies that show oral alcohol increases extracellular DA in brain reward circuitry (e.g., NAC) known to mediate drug reinforcement and NTX blocks this effect. The studies demonstrate that NTX decreases consummatory behaviors in rats of both sexes with a greater effect on consummatory responding in males in contrast to a greater effect on appetitive responding in females.

Effect of MG alone on alcohol self-administration. The present study is the first to determine the effects of MG on operant alcohol self-administration in rats. For example, one study used alcohol-preferring wild-type C57/BL6NHsd mice (male and female) and utilized a drinking-in the-dark binge protocol whereby only alcohol (10 and 20%) was made available intermittently and for a limited amount of time (4 hours). When a kratom extract, MG, and the alkaloid and purported MG metabolite 7-hydroxymitragynine (7-HMG) were administered prior to alcohol access, results showed administration of the kratom extract decreased intake in both male and female mice. Using a two-bottle choice (10% alcohol) procedure, MG (30 and 100 mg/kg, i.p.) significantly reduced alcohol consumption as did 7-HMG in mice of both sexes. These results are consistent with those from the present study, however; the doses of MG that decreased alcohol consumption in mice were much higher than the doses tested in rats in the present study (e.g., 0.3, 1 and 3.0 mg/kg, i.p.). Interestingly, and also consistent with this study, the highest dose of MG tested in the present study (3.0 mg/kg) decreased heroin self-administration in male Sprague Dawley rats.

Comparison of MG and NTX alone to its combination on alcohol self-administration. The combination of MG+NTX was more efficacious at decreasing alcohol self-administration than either drug alone, an unexpected finding. Decreases in three of the four self-administration measures including the appetitive measure of active lever presses and both measures of consummatory behavior (numbers of reinforcers earned and estimated total alcohol consumed) were more significant with the combination of the two drugs than what was seen for each drug alone (see Table 1 and FIG. 4). Specifically, the combination of MG+NTX had a significantly greater effect on estimated alcohol intake at the 0.3 mg/kg dose as well as showing some trends for greater effects in other measures and at other doses compared to MG alone. Compared to NTX alone, the combination had greater effects on three measures at the 3 mg/kg dose as well as a trend for a greater effect on active lever presses at the 1 mg/kg dose. It was also found that MG had more significant effects on all four measures of alcohol self-administration than NTX. Further, there was a trend for a greater effect of MG on active lever presses at the 3 mg/kg dose.

It is unlikely that the decrease in alcohol self-administration under any drug condition reflected a depression of activity levels. MG alone or when combined with NTX had no significant impact on total locomotor activity across 30-min sessions (FIGS. 5A & B) suggesting that its ability to reduce alcohol self-administration was not due to non-specific motor depression effects. In fact, MG and NTX alone tended to increase locomotor activity when assessed over 5-min time blocks compared to vehicle treatment. The lack of effect on total distance traveled across the session is consistent with a study using male Wistar rats following administration of a kratom extract (60 mg/kg, p.o.). However, some studies demonstrate that higher doses (10-30 mg/kg, ip) of MG but not lower doses (1, 5, 10 mg/kg, i.p.) increase locomotor activity in male Sprague Dawley rats. Studies in mice find no effect on spontaneous locomotor activity following high doses (50-500 mg/kg, p.o.) of a kratom extract or moderate to high doses (5-30 mg/kg, i.p.) of MG. In contrast, two studies using male mice found a significant decrease in activity following administration of a kratom extract containing MG+paynantheine (0.5+0.025 mg/kg) delivered p.o. and higher doses (2.0+0.1, 4.0+0.2 mg/kg) administered i.p. and MG administered alone (5, 10, 15 mg/kg, i.p.). Inconsistencies between studies may be due to differences in the type of kratom used or dose of MG, route of administration, mouse strain, test apparatus, time of session, or dosing regimen.

Effects of MG, NTX and combination on cFos expression. This is the first study to determine the central effects of MG, NTX and the MG+NTX combination by measuring cFos expression in various brain areas linked to alcohol-driven behaviors using immunohistochemistry (FIG. 6). cFos protein expression is mediated by the immediate early proto-oncogene cfos and increased levels are used as a non-specific marker of neuronal activation. The combination of MG+NTX induced greater cFos protein expression in many brain areas assessed than either drug administered alone (FIG. 6) mirroring that the combination was more robust at decreasing operant alcohol self-administration than either drug administered alone. The only exception was in the NAC where NTX alone increased cFos levels to a similar degree to that of MG and NTX combined (FIG. 6B).

How NTX increased cFos expression in the NAC is unknown, however there are opioid receptors in the NAC and the opioid agonist morphine is self-administered directly into this brain area. The NAC receives widespread excitatory afferents from the pre-frontal cortex. Depending on the location of G-protein linked mu opioid receptors, they can regulate neuronal excitability and are generally inhibitory within the NAC. Although speculative, NTX may block mu-receptor mediated inhibitory control within the NAC and enhance excitatory drive thereby increasing cFos activation. NTX-induced increases in NAC cFos found herein is consistent with a recent study showing the drug (2 mg/kg, i.p.) increases cFos expression in several brain areas including the NAC.

The dorsal striatum stores procedural memories, is involved in goal-directed behaviors, and controls habit learning. Whereas the NAC and its subregions mediate the primary reinforcing effects of alcohol and cue-controlled alcohol seeking, the dorsal striatum regulates habitual or compulsive alcohol-seeking behaviors that appears to be DA-dependent. As shown in FIG. 5C, and like the NAC and other areas (see below), a highly significant increase in cFos expression was observed following administration of MG+NTX together but not with either drug alone. How the combination increases cFos expression in the dorsal striatum is not clear, however MG induces DA release in other brain areas and NTX in combination with alcohol increases DA in the striatum.

The combination of MG and NTX also increased cFos levels in the IL CTX but not in the PL cortex (FIG. 5F). The IL cortex is a subregion of the medial pre-frontal CTX and regulates numerous behaviors including drug-seeking, reward, fear and extinction learning. The IL cortex also projects to the NAC, neural circuitry known to mediate drug reinforcement. Importantly, enhancing IL CTX activity decreases drug-seeking behavior whereas inactivation of the IL CTX increases cue-induced reinstatement of alcohol-seeking behavior. Thus, increases in cFos activity within the IL CTX following MG+NTX may have contributed to the robust decreases in oral alcohol self-administration measures seen in the study.

A significant increase in cFos expression in Cg1 and a trend towards a significant increase in Cg2 was observed in rats that received both MG+NTX but neither drug alone compared to controls (FIGS. 6H and I). The nomenclature used to describe the cingulate CTX across species has changed over time making comparisons between studies confusing however in general both Cg1 and Cg2 in the rat cover areas of what would be considered the mid-cingulate (MCC) and anterior cingulate cortex (ACC) in humans. Unlike humans that have distinct sub-regions within the cingulate CTX that mediate specific processes and functions such as regulation of autonomic responses, emotional and motivational aspects of external information, these functions seem to overlap across Cg1/Cg2 in the rat. Nevertheless, evidence indicates the cingulate CTX plays an important role in mediating alcohol-associated behaviors. Indeed, neurons located within the ACC and overall volume of this brain area are significantly reduced following chronic alcohol consumption in rats and infusion of a mu receptor antagonist into the ACC blocks cue-induced alcohol seeking behavior in mice. A human fMRI study shows abnormal connectivity between the various sub-regions of the cingulate CTX (ACC, MCC, posterior cingulate CTX) following exposure to alcohol and stress cues and this abnormal connectivity signature predicts relapse to alcohol drinking. Further, glutamatergic dysregulation within the ACC (and NAC) correlates positively with alcohol craving in recently detoxified individuals with AUDs. Accordingly, a small clinical trial showed that stimulating the ACC with bilaterally surgically implanted electrodes significantly decreased alcohol craving and consumption in heavy drinkers with AUD that were refractory to multiple treatment modalities. That the combination of MG+NTX increased activation within the cingulate CTX may relate to significant decreases in alcohol consumption seen in the present study.

The fact that rats were exposed to a novel locomotor apparatus 90-min prior to obtaining brain tissue samples for the cFos immunohistochemistry study may have influenced cFos levels particularly in the dorsal striatum that mediates motor control. However, cFos was only significantly elevated in the group that received the MG+NTX combination and either drug administered alone did not differ from the control group. Thus, it is most likely that the increased neural activity in the dorsal striatum was due to administration of MG+NTX. It must be noted also that rats used in the immunohistochemistry experiment were not exposed to alcohol which could impact the cFos expression profiles. Future studies should assess the effects of MG, NTX and MG+NTX in rats that have been exposed to alcohol.

Potential mechanisms of action for MG. Contradictory evidence suggests that MG may act as a G-protein biased mu receptor agonist, partial agonist, antagonist at delta opioid receptors or “does not directly activate opioid receptors”. Results presented here do not support this since NTX did not attenuate MG's effects on any measure of operant alcohol self-administration. Our results are however consistent with a study by Hiranita et al. showing NTX (1 mg/kg) did not block MG's ability to decrease schedule-controlled responding. In fact, the MG+NTX combination decreased alcohol self-administration to an even greater degree than MG alone (FIGS. 1, 3, and 4; Table 1). The enhanced effects of MG+NTX suggests MG may be acting as an antagonist at mu receptors although MG does not block the analgesic effects of morphine. Our results also do not support the idea that MG elicits partial agonist activity at mu opioid receptors at least within the context of operant alcohol self-administration related behaviors. While some research shows that opioid agonists enhance alcohol consumption, other studies find decreased alcohol effects consistent with the results with MG in the present study.

MG has similar binding affinities at mu and kappa opioid receptors as measured by in vitro displacement studies suggesting that kappa receptors may be an alternative mechanism of action for reducing alcohol self-administration. However, in addition to mu opioid receptors, NTX is also an antagonist at kappa opioid receptors and binding to this receptor is associated with reduced alcohol drinking and alcohol craving in humans. Presumably, NTX would also be expected to block any potential activity of MG at the kappa opioid receptor subtype. Compounds targeting delta opioid receptors, in particular, antagonists, decrease alcohol related behaviors. Indeed, MG has activity at delta opioid receptors as measured by G-protein-mediated inhibition assays. MG also inhibits forskolin-stimulated CAMP in a concentration manner in cells (NG108-15) that possess delta opioid receptors and this effect is blocked by naloxone. Other evidence however demonstrates very low binding to delta opioid receptors by MG. Nevertheless, the ability of MG to decrease alcohol self-administration most likely depends upon neurotransmitter systems other than the opioid system.

Potential therapeutic use of MG for treating AUDs. That MG has been shown to induce place conditioning and partially substitute for morphine in drug discrimination tests may raise concerns of abuse liability; however, these effects were generated using significantly higher doses (15-30 mg/kg) than those used in the present study. Yet, an intracranial self-stimulation (ICSS) study did not find evidence that MG (3.2-56 mg/kg) was rewarding in male or female rats. The gold-standard behavioral paradigm to detect abuse liability is whether the compound is self-administered by animals. When a broad range of MG doses were substituted in rats trained to self-administer (i.v.) morphine, it did not support self-administration. MG (0.03-3.0 mg/kg) also did not maintain responding above saline levels when substituted for heroin and, in fact, actually decreased heroin self-administration in rats. Interestingly, prior exposure to MG decreases i.v. morphine self-administration consistent with the finding that individuals use kratom to treat their OUD. In contrast, the alkaloid 7-HMG, also contained in kratom, was readily self-administered by rats although its abuse liability was not shown in ICSS. Doses of MG (3.0 mg/kg) that decreased heroin self-administration also decreased alcohol self-administration as demonstrated in the present study. Overall, these data suggest MG does not possess significant abuse potential at doses that decrease alcohol self-administration. Thus, MG alone or in combination with the FDA-approved NTX may be a beneficial treatment for AUDs although these results should be extended to male rats.

Conclusions. MG alone significantly decreased all alcohol self-administration measures and reduced total alcohol consumed. NTX also decreased most of these same measures however in a less robust manner. Surprisingly, administration of MG+NTX combination resulted in further decreases in self-administration measures to a greater degree than either compound administered alone. These effects were not due to adverse effects of MG+NTX as demonstrated by results from the open field test. Further, cFos expression from several brain areas implicated in alcohol reinforcement and consumption mirrored the effects seen on alcohol self-administration. That is, the MG+NTX combination was associated with greater cFos expression than when either of the compounds were administered alone. Taken together, these data strongly suggest that MG is likely impacting non-opioid neurotransmitter systems involved in alcohol reinforcement.

What is claimed is:

1. A therapeutic drug composition, comprising:
mitragynine; and
naltrexone.

2. The therapeutic drug composition of claim 1, wherein the therapeutic drug composition comprises equal concentrations of mitragynine and naltrexone.

3. The therapeutic drug composition of claim 1, wherein the therapeutic drug composition comprises concentrations of mitragynine and naltrexone in a ratio of mitragynine to naltrexone of between 1:5 and 1:10.

4. The therapeutic drug combination of claim 1, comprising a therapeutically effective amount of mitragynine and naltrexone, and excluding other active ingredients.

5. The therapeutic drug composition of claim 1, comprising a therapeutically effective amount of mitragynine and naltrexone, and further comprising a pharmaceutically acceptable excipient, adjuvant, carrier, buffer, stabilizer, or mixture thereof.

6. The therapeutic drug composition of claim 5, wherein the therapeutic drug composition comprises 11.35 mg to 33.6 mg of mitragynine and 11.35 mg to 33.6 mg naltrexone.

7. The therapeutic drug composition of claim 5, wherein the therapeutic drug combination comprises 39 mg to 50 mg of mitragynine and 39 mg to 50 mg of naltrexone.

8. A method for treatment of substance use disorders comprising the step of administering the therapeutic drug composition of claim 5 to a subject having a substance use disorder.

9. The method of claim 8, wherein the substance use disorder is alcohol use disorder or opioid use disorder.

10. A method for reducing self-administration of alcohol by a subject comprising the step of administering the therapeutic drug composition of claim 5 to the subject.

11. The method of claim 8, wherein the therapeutic drug composition comprises 11.35 mg to 33.6 mg of mitragynine and 11.35 mg to 33.6 mg naltrexone, and wherein the subject has a weight of 70 kg to 80 kg.

12. The method of claim 8, wherein the therapeutic drug composition comprises 39 mg to 50 mg of mitragynine and 39 mg to 50 mg of naltrexone, and wherein the subject has a weight of 80 kg to 90 kg.

13. The method of claim 10, wherein the therapeutic drug composition comprises 11.35 mg to 33.6 mg of mitragynine and 11.35 mg to 33.6 mg naltrexone, and wherein the subject has a weight of 70 kg to 80 kg.

14. The method of claim 10, wherein the therapeutic drug composition comprises 39 mg to 50 mg of mitragynine and 39 mg to 50 mg of naltrexone, and wherein the subject has a weight of 80 kg to 90 kg.
